(19) United States
(12) Patent Application Publication
(10) Pub. No.: US 2015/0369801 A1
(43) Pub. Date: Dec. 24, 2015

(54) Method for Detecting and/or Quantifying the Binding Affinities of a Target Molecule to a Plurality of Different Binding Partners by Plasmon Resonance of Nanoparticles and a Position-Encoded Sensor Therefor

(71) Applicant: Carsten Sönnichsen, Mainz (DE)

(72) Inventors: Carsten Sönnichsen, Mainz (DE); Christina Lambertz, Neuwied (DE); Rubén Ahijado-Guzmán, Mainz (DE); Janak Prasad, Mainz (DE)

(21) Appl. No.: 14/310,208

(22) Filed: Jun. 20, 2014

(51) Int. Cl. G01N 33/543; G01N 21/65

(52) U.S. Cl. CPC G01N 33/54353; G01N 33/54386; G01N 21/658

(57) ABSTRACT

A method for detecting and/or quantifying the binding affinities of one sort of target molecule to a plurality of different binding partners includes using plasmon resonance of nanoparticles, and a position-encoded sensor to measure shifts in the plasmon resonance of the nanoparticles when the target molecule is bound thereto.

### FIELD OF THE INVENTION

The present invention relates to a method for detecting and/or quantifying the binding affinities of a target molecule to a plurality of different binding partners using the plasmon resonance of nanoparticles in conjunction with a position-encoded sensor.

### BACKGROUND OF THE INVENTION

The dynamics in living organisms are governed by a complex network of interacting macromolecules. For example, in a process like cell division, several different proteins interact in a subtle and interconnected way of sub-processes to finally define a division place or to generate the constriction force in a cell wall. To fully understand, model and potentially influence such a process by drugs, it is important to detect and/or quantify binding affinities between all possible partners, preferably without introducing fluorescent labels. Popularly techniques to do so include surface plasmon resonance (SPR) biosensors, quartz crystal microbalance, fluorescence correlation spectroscopy, isothermal titration calorimetry, or analytical ultracentrifugation. However, most of the label-free techniques used to study binding affinities require the analysis of one pair of binding partners at a time, making the quantification of a complex interaction network a laborious and slow process. While there are some approaches on the market to increase the throughput by effectively building several experiments into one device, e.g. multi-flow-channel SPR chips, these approaches are expensive compared and not easily up-scalable.

Therefore, it is desired to provide a method to detect and/or quantify binding affinities between multiple molecular partners in a single experiment thereby increasing the throughput.

### SUMMARY OF THE INVENTION

One aspect of the present invention includes a method for detecting and/or quantifying binding affinities. The method includes the steps of providing batches of plasmonic nanoparticles, wherein each batch of the batches of plasmonic nanoparticles is functionalized with a different binding partner. After each of the batches of plasmonic nanoparticles are provided and functionalized, each batch is deposited consecutively on a carrier substrate in a random manner and the position of each batch of functionalized plasmonic nanoparticles is recorded on the carrier substrate after each deposition to define a position-encoded carrier substrate. A target molecule is then introduced to the position-encoded carrier substrate and is allowed to bind to specific binding partners of the different binding partners which interact with the target molecule. This binding interaction between specific binding partners and the target molecule induces a shift in a plasmon resonance of the functionalized plasmonic nanoparticles. The shift in the plasmon resonance is then measured.

Another aspect of the present invention includes a method for detecting and quantifying binding affinities of a target molecule to different binding partners. The method includes the steps of providing batches of plasmonic nanoparticles, wherein each batch of plasmonic nanoparticles is functionalized with a different binding partner. Each batch is then deposited consecutively on a carrier substrate in a random manner. The position of each functionalized plasmonic nanoparticle on the carrier substrate is then recorded after each batch deposition to provide a position-encoded carrier substrate. A first spectral signature for each batch of functionalized plasmonic nanoparticles deposited on the carrier substrate is then determined. A target molecule is then introduced to the position-encoded carrier substrate and allowed to bind to specific binding partners which interact with the target molecule, thereby inducing a shift in plasmon resonance of the plasmonic nanoparticles functionalized with the specific binding partners. The shift in the plasmon resonance from the first spectral signature to the second spectral signature is then measured.

Yet another aspect of the present invention includes a position-encoded sensor for plasmon resonance-based detection and/or quantification of binding affinities of a target molecule to a plurality of different binding partners. The position-encoded sensor comprises a carrier substrate including a surface. A plurality of differently functionalized plasmonic nanoparticles are disposed randomly on the surface of the carrier substrate, and the position of each differently functionalized plasmonic nanoparticle is recorded to define a position-encoded sensor.

### BRIEF DESCRIPTION OF THE DRAWINGS

In the drawings:

FIG. 1A is a graphical representation of a carrier substrate fabrication strategy according to one embodiment of the present invention;

FIG. 1B is a graphical representation of a binding affinity detection method using the carrier substrate of FIG. 1A;

FIG. 2A is a graphical representation of a functionalization process for plasmonic nanoparticles using various binding partners;

FIG. 2B is a graphical representation of a carrier substrate fabrication strategy according to another embodiment of the present invention;

FIG. 2C is a graphical representation of a binding affinity detection method using the carrier substrate of FIG. 2B;

FIG. 3A is graphical representation mapping shifts in wavelength of plasmonic nanoparticles functionalized with a first protein relative to the concentration of a target molecule on a carrier substrate;

FIG. 3B is graphical representation mapping shifts in wavelength of plasmonic nanoparticles functionalized with a second protein relative to the concentration of a target molecule on a carrier substrate;

FIG. 3C is graphical representation mapping shifts in wavelength of plasmonic nanoparticles functionalized with a third protein relative to the concentration of a target molecule on a carrier substrate; and

FIG. 3D is a graphical representation mapping shifts in wavelength of non-functionalized plasmonic nanoparticles relative to the concentration of a target molecule on a carrier substrate.

### DETAILED DESCRIPTION OF THE PREFERRED EMBODIMENTS

The present invention provides a simple, fast and cost-effective method to detect and/or quantify the binding affinities of a target molecule to a plurality of different binding partners by using individually functionalized plasmonic nanoparticles as sensing elements. Each plasmonic nanoparticle responds to binding events near its surface by a change in the plasmon resonance shifting the plasmon resonance wavelength. The shift in the plasmon resonance can be detected on individual particles. As used herein, the term “and/or,” when used in a list of two or more properties of a method, means that any one of the listed properties can be determined using a method either alone, or in combination of two or more of the listed properties. For example, if a method is described as determining properties A, B, and/or C, then the method can be used to determine property A alone; property B alone; property C alone; property A and property B in combination; property A and property C in combination; property B and property C in combination; or properties A, B, and C in combination.

The plasmonic nanoparticles of the present invention refer to particles with at least one dimension, preferably two or three dimensions, of less than 1 μm, preferably 500 nm, more preferably 300 nm. Embodiments may have all dimensions less than 1 μm and at least one dimension less than 100 nm. The plasmonic nanoparticles are fine metal particles, preferably of gold or silver, and preferably have the shape of sphere-capped cylinders. The aspect ratio is preferably above 2, more preferred between 2.5 and 4.5, most preferred between 3 and 4 and the width is preferably between 20 and 45 nm. In a preferred embodiment, the plasmonic nanoparticles are gold particles which are rod-shaped, hollow shells or shaped like bipyramids.

The binding partner(s) (P₁-Pᵢ) may be any kind of molecule, such as antibodies, antigen binding fragments of antibodies, peptides, proteins, antigens, polynucleotides, oligonucleotides, peptide nucleic acids or artificial binding agents, which are specific for a certain binding substance (target). Thus, any kind of target can be detected.

A target molecule does not refer to one target molecule but refers to one single type of target molecules which are used in the method according to the present invention. The target molecule (T) is preferably a biological macromolecule such as a nucleic acid, a protein, a carbohydrate, a lipid or a synthetic macromolecule such as a simple polymer, a dendrimer or a di-block-copolymer. The target molecule has preferably a size of 2 to 100 nm, preferably 5 to 10 nm, wherein the size corresponds to the diameter in the longest dimension. In particular, the target molecules may be polypeptides or proteins having a molecular weight between 1 and 3000 kDa, preferably between 20 kDa and 300 kDa.

In the method of the present invention the plasmonic nanoparticles are functionalized with different types of binding partner P_{1 to} Pᵢ. This functionalization is carried out in two steps. First, a selected ligand, e.g. Ni-NTA, is coupled to all plasmonic nanoparticles thereby creating a stock solution. The stock solution can be divided into different batches or aliquots, preferably provided in different tubes, vessels or wells. Then, in a second step, a first binding partner (P₁) having a tag corresponding to the selected ligand, e.g. a His-tagged protein, is added to the first batch of the ligand-coupled plasmonic nanoparticles. Thereby the binding partner P₁ is coupled to the plasmonic nanoparticles. The second step is repeated with a further batch of ligand-coupled plasmonic nanoparticles, i.e. the second batch, and a second binding partner (P₂) having a tag corresponding to the selected ligand. Thereby the binding partner P₂ is coupled to the plasmonic nanoparticles. Accordingly, it is possible to create a library of differently functionalized plasmonic nanoparticles having binding partners P₁ to Pᵢ with relative ease and, for example, in an automated system using multi-well plates. Furthermore, it is possible to simply mix a drop of the stock solution with one of the binding partner P₁ to Pᵢ in a small reaction vessel.

By this functionalization, the plasmonic nanoparticles of the present invention are connected to the different proteins (P₁ to Pᵢ) via specific linkers comprising a ligand and a corresponding tag. The ligands are selected from commonly known ligands, such as Ni-NTA, strepavidin, avidin, antigen and glutathione, and the corresponding tags are selected from commonly known tags, such as polyhistidin-tag (His-tag), Strep-tag, biotin-tag, antibody-tag and GST-tag. It is important that the linker does not interfere with the function of the binding partner.

It is also necessary that the functionalized plasmonic nanoparticles provide sufficient stability in buffers commonly use in biological setting and do not aggregate. Therefore, to create the stock of ligand-coupled plasmonic nanoparticles, it is usually necessary to stabilize them. In the present invention this is achieved by replacing the initial stabilizers of the plasmonic nanoparticles (a surfactant) by shell of bi-functional small polymers, specifically bifunctional DNA strands consisting of, for example, eleven thymidine bases with a thiol and an amine endgroup at the DNA ends. The thiol group can readily bind to the plasmonic nanoparticles whereas the amine group specifically couple to the ligand, e.g. to Ni-NTA, that later links to a tag on the binding partners P₁ to Pᵢ.

This process is further improved, in the sense of less aggregates forming, by first using only mono-functional stabilizers that attach to the plasmonic nanoparticles and then filling remaining gaps on the nanoparticle surface with bifunctional stabilizers.

Therefore, before the functionalization step, the plasmonic nanoparticles are stabilized preferably with short bifunctional DNA strands, which preferably consisting of eleven thymidine bases and a thiol group at the end.

In a next step, the functionalized plasmonic particles from the above-mentioned library are consecutively deposited on a carrier substrate in a random manner. As a carrier substrate, any support may be used, e.g. solid supports like glass, resins, plastics, metal, films and/or gels. The carrier substrate can be arranged within a flow cell. It is necessary that each batch (1 to i) of the functionalized plasmonic nanoparticles is deposited separately while recording the position of each randomly deposited nanoparticle. Accordingly, it is possible to know the specific functionalization for each particle on the carrier substrate, thus effectively generating a position-encoded carrier substrate, similar to a spotted array but without any geometrical order. The position of each plasmonic nanoparticle can be recorded by dark field microscopy, reflection microscopy with crossed polarizers, total internal reflection microscopy or fluorescence microscopy.

To evaluate the binding affinities of one type of target molecules to the different binding partners, the position-encoded carrier substrate is exposed to the target molecules (T). The introduction of target molecules can also be the flow of the target molecules over the carrier substrate as, for example, carried out in a flow cell. If the target molecule binds to one or multiple of the binding partners (P₁ to Pᵢ), a shift Δλᵣₑₛ in the plasmon resonance of the corresponding nanoparticles is induced. In particular, the carrier substrate may be exposed to a solution with increasing target concentration similar to the titration method described by Schuck (see Analytical Biochemistry, Schuck, P. et al. 1998, 265, (1), 79-91). Then the wavelength shift Δλᵣₑₛ is recorded for each nanoparticle on the carrier substrate as described in the International patent application WO 2004/042403, incorporated herein by reference in its entirety. The recorded wavelength shifts for each group of nanoparticles (each group corresponding to particles functionalized with one binding partner P) provide, via the above mentioned titration method, the binding affinities of the target molecule T to each of the binding partners P₁ to Pᵢ.

Further, the present invention relates to a position-encoded sensor which is characterized by a sensor surface comprising a plurality of differently functionalized plasmonic nanoparticles which are deposited on the sensor surface in a random manner, that is to say, without any geometrical order. This random deposition simplifies the production of the sensor by avoiding complex and unnecessary fabrication steps such as “blotting”. The random deposition results in a very specific sensor surface with elements, such as plasmonic nanoparticles, recording bindings of the target molecule T specifically to several different binding partners P₁ to Pᵢ within a very small sensor area, typically 100 μm by 100 μm. It is clear that the functionalized plasmonic nanoparticles deposited on the position-encoded sensor are characterized by the same features as already mentioned for the method according to the present invention.

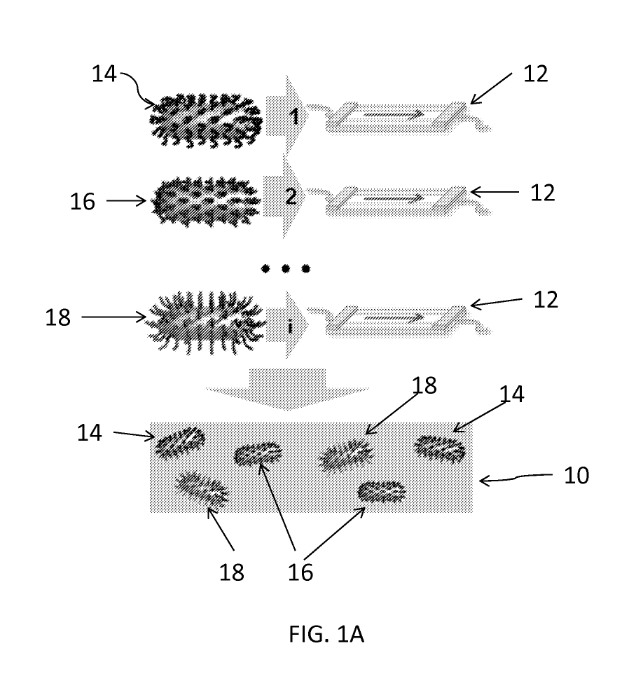

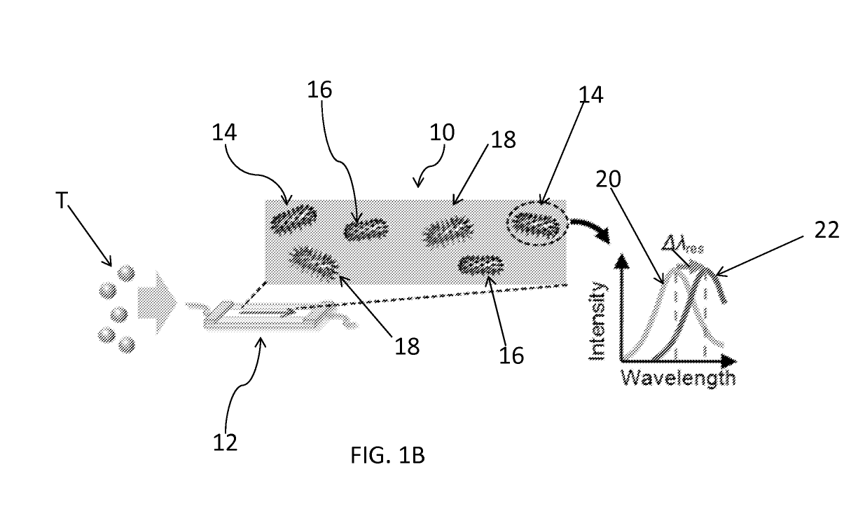

Referring now to FIGS. 1A-3D, in FIG. 1A a mapped or position-encoded carrier substrate 10 is shown, which may also be referred to herein as a sensor. The position-encoded carrier 10 is fabricated as each batch of plasmonic nanoparticles 14-18, which are each functionalized with different binding partners P₁ to Pᵢ, is deposited consecutively as indicated by arrows 1 to i in a microfluidic flow cell 12. In the embodiment of FIG. 1A, batch 14 of plasmonic nanoparticles is functionalized with binding partner P₁. Batch 16 of plasmonic nanoparticles is functionalized with binding partner P₂. Batch 18 of plasmonic nanoparticles is functionalized with binding partner Pᵢ. As the batches 14-18 are consecutively deposited on the carrier 10 using the microfluidic flow cell, the position of each batch 14-18 is recorded after each batch deposition. Thus, the position of the deposits of batches 14, 16 and 18 are recorded in the resulting position-encoded carrier 10.

Referring now to FIG. 1B, a target molecule T is introduced to the flow cell 12 by being injected into the flow cell 12. The target molecule T specifically binds to its corresponding binding partner(s) P₁ to Pᵢ as found on plasmonic nanoparticles 14-18, and induces a shift Δλᵣₑₛ in plasmon resonances from a first spectral signature 20 to a second spectral signature 22. The first spectral signature 20 of FIG. 1B indicates a wavelength consistent with plasmonic nanoparticles 14 before interaction with the target molecule T. The second spectral signature 22 of FIG. 1B indicates a wavelength consistent with plasmonic nanoparticles 14 after interaction with the target molecule T, which may include binding of the target molecule T to binding partner P₁. After determining the first and second spectral signatures 20, 22, the difference in the wavelength between the first and second spectral signatures 20, 22 can be measured and given a value Δλᵣₑₛ. As used herein, the term “spectral signature” identifies a charted measure of an intensity of SPR reflectivity represented as a function of wavelength.

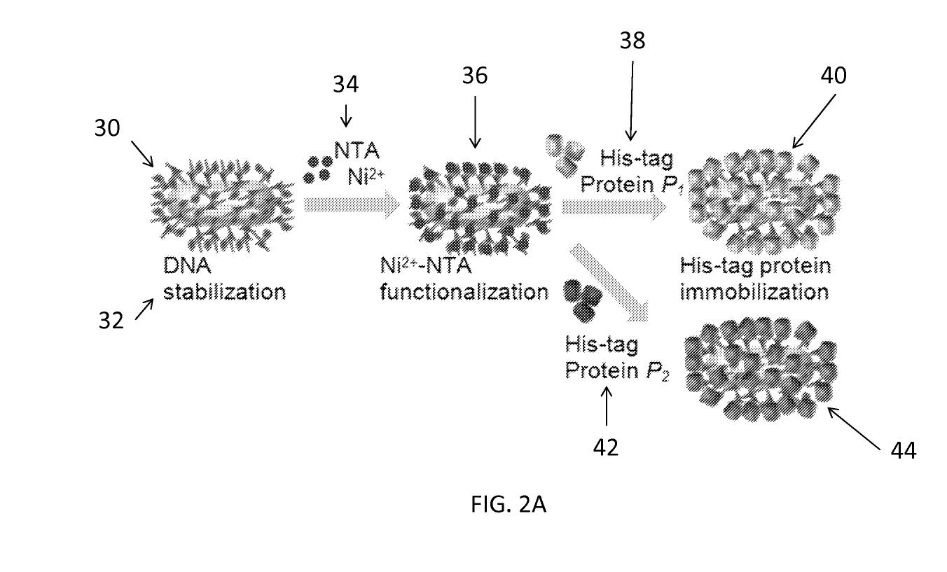

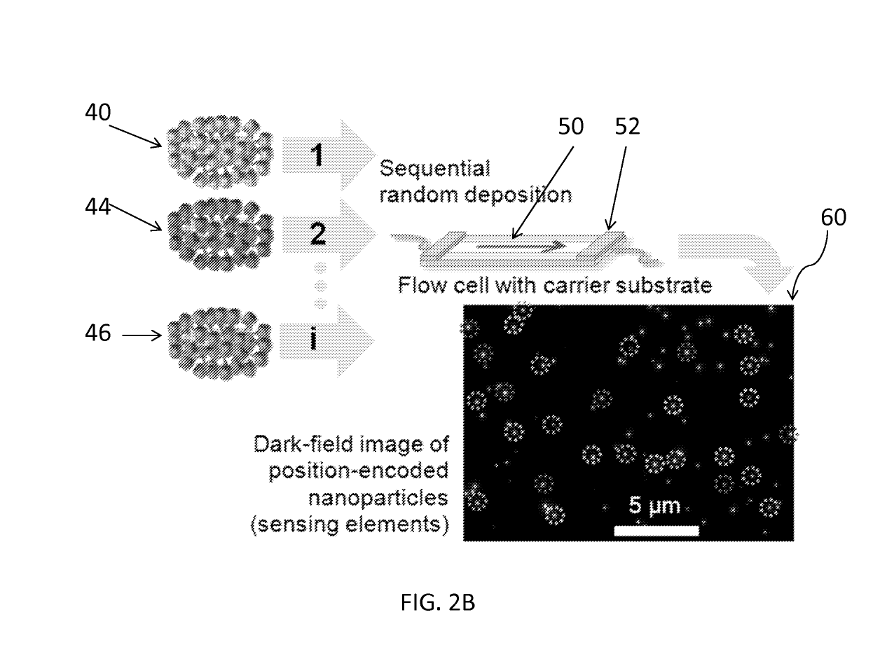

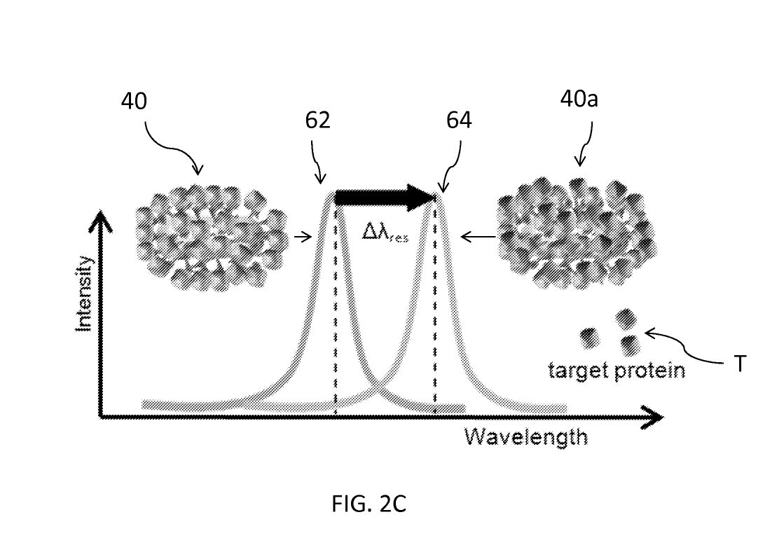

Referring now to FIG. 2A, gold nanoparticles 30 are first stabilized with DNA in a step indicated by reference numeral 32. Afterwards the gold nanoparticles 30 are functionalized in a two-step process. First, Ni-NTA is coupled to the gold nanoparticles as indicated by reference numeral 34 to create Ni-NTA gold nanoparticles 36. Then, different batches of the Ni-NTA gold nanoparticles 36 are incubated with different His-tagged proteins P₁, P₂ and Pᵢ. Accordingly, differently functionalized gold nanoparticles are achieved. As shown in FIG. 2A, Ni-NTA gold nanoparticles 36 are shown incubated with His-tagged proteins P₁, P₂ in steps 38 and 42 to form functionalized gold nanoparticles 40 and 44 respectively. Then each batch (1 to i) of functionalized gold nanoparticles 40, 44 and 46, wherein functionalized gold nanoparticles 46 are functionalized with His-tagged proteins Pᵢ, is consecutively deposited on a carrier substrate 50 using a flow cell 52, as shown in FIG. 2B. After each deposition step, represented by arrows 1, 2 and i, the position of the functionalized gold nanoparticles 40, 44 and 46 is recorded, thereby creating a position-encoded sensor 60. The inset image of the encoded sensor 60 shown in FIG. 2B shows a dark-field image of the resulting randomly deposited gold nanoparticles 40, 44 and 46. Each nanoparticle serves as a sensing element for a specific binding partner-target interaction which is visualized by the dashed circles in FIG. 2B. The binding of the target protein T injected into the flow cell to nanoparticles functionalized with the different proteins (P₁ to Pᵢ) produces a shift Δλᵣₑₛ in the plasmon resonance, as further shown in FIG. 2C. In FIG. 2C, functionalized gold nanoparticles 40 is shown having a first spectral signature 62. After interaction, such as binding, with target molecule T to form nanoparticle 40a, a second spectral signature 64 is demonstrated. After determining the first and second spectral signatures 62, 64, the difference in the wavelength between the first and second spectral signatures 62, 64 can be measured and given a value Δλᵣₑₛ.

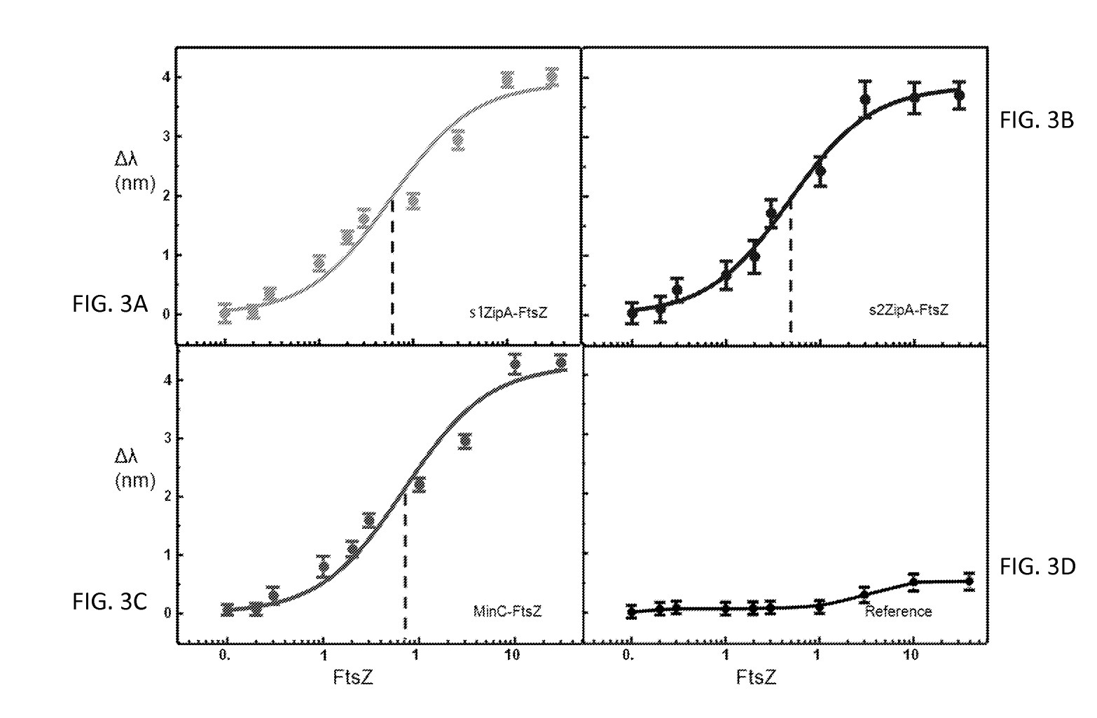

Referring now to FIGS. 3A-3D, the results of Example 1, detailed below, are shown, wherein plasmonic nanoparticles are functionalized with s1-ZipA (FIG. 3A), s2-ZipA (FIG. 3B), or MinC (FIG. 3C). The data-points in FIGS. 3A-3D correspond to the mean plasmon shift Δλᵣₑₛ measured on 30 to 50 nanoparticles. The error bar indicates the standard error of the mean. The plasmon shifts Δλᵣₑₛ are measured relative to increasing concentrations of the target protein FtsZ (after equilibration) and obtained on a single Nano-SPR sensor. The solid lines correspond to the best fit of a Langmuir equation to the experimental data used to extract the K_{D} values listed in Table 1 and shown in dashed lines in FIGS. 3A-3D.

### Example 1

In an embodiment four batches of gold nanorods were deposited on a sensor substrate (carrier substrate). The first batch of nanorods was functionalized with protein P₁ (s1-ZipA) (FIG. 3A), the second batch was functionalized with protein P₂ (s2-ZipA) (FIG. 3B) (s1-ZipA and s2-ZipA lack its transmembrane domain), the third batch was functionalized with protein P₃ (MinC) (FIG. 3C), and the fourth batch was not functionalized at all and serves as reference or negative control for absent interaction (FIG. 3D).

Firstly, the four batches of gold nanorods were stabilized with T-linker DNA (5′-SH-TTTTTTTTTTT-3′) and Thiolated DNA (5′-SH-TTTTTTTTTTT-NH₂-3′). Then the nanorods were reacted with isothiocyanobenzyl-NTA in suspension. At last, the nanorods were incubated with NiCl₂.

Then, three of the four batches of gold nanorods were incubated with their respective His-tagged proteins P₁, P₂ and P₃ (s1-ZipA, s2-ZipA and MinC). By UV-Vis NIR spectra it was clearly visible that the proteins were attached to the nanorods (characteristic resonance of the protein 280 nm).

Each of the four batches was separately flowed over the sensor substrate while the position of each randomly deposited nanoparticle was recorded. Therefore, it was possible to know the specific functionalization for each particle on the sensor surface, thus effectively generating a position-encoded sensor, similar to a spotted array but without geometrical order.

Then the target protein T (FtsZ, a 40 kDa soluble GTPase, homolog of eukaryotic tubulin) was flushed through the flow cell. In Escherichia coli, FtsZ is anchored to the cytoplasmatic membrane by the action of the membrane protein ZipA and the amphitropic protein FtsA.

The plasmon shift is recorded by optical dark-field spectroscopy for each single gold nanorod sensor in the field of view using a fully automated, temperature stable, user friendly and fast optical microscopic system optimized for spectral precision. This system allows routinely recording the response (spectral shift of plasmon resonance) of hundreds of particles with a precision of about 0.3 nm.

To evaluate the binding affinities of the three interacting pairs (s1ZipA-FtsZ (FIG. 3A), s2ZipA-FtsZ (FIG. 3B), and MinC-FtsZ (FIG. 3C)), the sensor was exposed to solutions with increasing FtsZ-concentrations, similar to the titration method of Schuck (see Analytical Biochemistry, Schuck, P. et al. 1998, 265, (1), 79-91). FtsZ aliquots with increasing concentrations were flowed into the flow cell until the interaction with the sensor reached equilibrium after about 20 minutes. The wavelength shift Δλᵣₑₛ of about 200 particles was recorded for each aliquot providing about 30 to 50 data for each interaction pair. FIGS. 3A-3D shows the average plasmon shift Δλ and the standard error of the mean as a function of FtsZ concentration, i.e. the binding isotherms, for each of the three interacting pairs and the non-functionalized reference particles. The flat response of the reference nanoparticles demonstrates that unspecific interaction between the sensors and FtsZ is negligible. The experimental data are well-described by the Langmuir equation Δλ/Δλₘₐₓ=cK_{D}/(1+cK_{D}), which allows to extract the equilibrium dissociation constants K_{D} for each pair. The K_{D} values for the three interacting pairs are listed in Table 1.

Table 1 shows the log K_{D} values obtained by Nano-SPR according to the present invention and SPR together with literature values determined by composition gradient-static light scattering (GC-SLS) (see Biochemistry Martos A. et al. 2010, 49, (51) 10780-7), sedimentation equilibrium-analytical centrifigaction (SE-AUC) (see Biochemistry Martos A. et al. 2010, 49, (51) 10780-7 and The Journal of biological chemistry Hernández-Rocamora, V. M. et al. 2012, 287, (36), 30097-104) and fluorescence anisotropy (FA) (see The Journal of biological chemistry Hernández-Rocamora, V. M. et al. 2013, 288 (34) 24625-35).

TABLE 1

| | Nano-SPR | SPR | GC-SLS | SE-AUC | FA |
|---|---|---|---|---|---|
| FtsZ-s1ZipA | 5.2 ± 0.2 | 5.4 ± 0.1 | 5.8 ± 0.1 | 5.4 | |
| FtsZ-s2ZipA | 5.3 ± 0.1 | 5.3 ± 0.2 | | | |
| FtsZ-MinC | 5.1 ± 0.1 | 5.0 ± 0.1 | | | 4.9 ± 0.1 |

The values of the Nano-SPR method are in excellent agreement compared with the other techniques. In contrast to all other techniques, Nano-SPR results according to the present invention were obtained on a single sensor providing binding affinities for all three proteins (P₁, P₂, P₃) and the reference in a single experiment.

Accordingly, it is possible to see the advantages of Nano-SPR to conventional SPR: the nanoscopic dimension of the sensing elements (1) drastically reduces the problem of non-uniform surface functionalization across the SPR sensor surface, (2) drastically reduces the required sample volume, (3) provides a build-in statistics without the need to repeat the measurements, (4) allows more easily to study many interaction pairs in a single experiment and (5) is fully compatible with automated liquid handling systems such as multi-well titration equipment. These advantages make Nano-SPR a simple, fast and cost-effective route to characterize binding affinities between multiple molecular partners. Accordingly, the claimed method can be used in analysis, drug screening, drug candidate evaluation, clinical diagnostics, veterinary diagnostics, pharmacology, environmental analysis, biophysics and/or biochemistry.

It will be understood by one having ordinary skill in the art that the methodology of the described invention is not limited to any specific technique. Other exemplary embodiments of the invention disclosed herein may be carried out using a wide variety of techniques, unless otherwise described herein.

It is also important to note that the construction and arrangement of the method steps of the invention as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present invention. The exemplary structures and processes disclosed herein are for illustrative purposes and are not to be construed as limiting.

It is also to be understood that variations and modifications can be made on the aforementioned structure without departing from the concepts of the present invention, and further it is to be understood that such concepts are intended to be covered by the following claims unless these claims by their language expressly state otherwise.

What is claimed is:

1. A method for detecting and quantifying binding affinities, the method comprising the steps of:
a) providing batches of plasmonic nanoparticles, wherein each batch of the batches of plasmonic nanoparticles is functionalized with a different binding partner;
b) depositing each batch of the functionalized plasmonic nanoparticles consecutively on a carrier substrate in a random manner;
c) recording the position of each functionalized plasmonic nanoparticle on the carrier substrate after each batch deposition to create a position-encoded carrier;
d) introducing a target molecule to the position-encoded carrier and allowing the target molecule to bind to specific binding partners which interact with the target molecule, thereby inducing a shift in a plasmon resonance of the plasmonic nanoparticles functionalized with the specific binding partners; and
e) measuring the shift in the plasmon resonance.

2. The method according to claim 1, wherein the binding partner is a binding partner selected from the group consisting of a protein, a peptide, an antigen, an antibody and DNA.

3. The method according to claim 2, wherein the target molecule is a target molecule selected from the group consisting of a nucleic acid, a protein, a carbohydrate, a lipid and a synthetic polymer.

4. The method according to claim 1, wherein the step of providing batches of plasmonic nanoparticles further includes the plasmonic nanoparticles being functionalized by:
coupling a ligand to the plasmonic nanoparticles; and
providing a tagged binding partner, wherein a tag of the tagged binding partner is configured to couple to the ligand.

5. The method according to claim 4, wherein the ligand is a ligand selected from the group consisting of Ni-NTA, strepavidin, avidin, antigen and glutathione.

6. The method according to claim 5, wherein the tagged binding partner is a tagged binding partner selected from the group comprising polyhistidin-tag, Strep-tag, biotin-tag, antibody-tag, GST-tag.

7. The method according to claim 1, wherein the step of introducing a target molecule further includes:
flowing a target molecule over the carrier substrate in a flow cell.

8. The method according to claim 1, wherein in the step of recording the position of each functionalized plasmonic nanoparticle on the carrier substrate is recorded using a technique selected from the group consisting of dark field microscopy, reflection microscopy with crossed polarizers, total internal reflection microscopy and fluorescence microscopy.

9. The method according to claim 4, wherein the step of providing batches of plasmonic nanoparticles further includes providing batches of plasmonic nanoparticles stabilized with short polymers.

10. The method according to claim 9, wherein the short polymers include short bifunctional DNA strands having first and second ends, wherein the first end of each DNA strand binds to the plasmonic nanoparticle, and the second end of each DNA strand is coupled to the ligand.

11. The method according to claim 10, wherein the bifunctional DNA strands consist of eleven thymidine bases and a thiol group disposed at the first and second ends.

12. The method according to claim 10, wherein the step of providing batches of plasmonic nanoparticles stabilized with short polymers further includes:
stabilizing the plasmonic nanoparticles with DNA strands having a thiol-group at one of the first and second end; and
replacing the DNA strands with bifunctional DNA strands having eleven thymidine bases and a thiol group disposed at the first and second ends.

13. The method according to claim 1, wherein the functionalized plasmonic nanoparticles are stable in solution.

14. The method according to claim 1, wherein the shift in the plasmon resonance is measured as a function of a concentration of the target molecule.

15. The method according to claim 1, wherein the step of providing batches of plasmonic nanoparticles further includes:
coupling a ligand to plasmonic nanoparticles to produce a stock solution; and
dividing the stock solution into different batches of plasmonic nanoparticles and linking each batch of plasmonic nanoparticles to different binding partners, wherein each binding partner has a tag that is able to couple to the ligand.

16. The method according to claim 1, wherein the plasmonic nanoparticles include gold particles.

17. The method according to claim 16, wherein the gold particles are gold particles selected from the group consisting of rod-shaped gold particles, hollow shell gold particles and bipyramid-shaped gold particles.

18. A method for detecting and quantifying binding affinities of a target molecule to different binding partners, the method comprising the steps of:
a) providing batches of plasmonic nanoparticles, wherein each batch of plasmonic nanoparticles is functionalized with a different binding partner;
b) depositing each batch of the functionalized plasmonic nanoparticles consecutively on a carrier substrate in a random manner;
c) recording the position of each functionalized plasmonic nanoparticle on the carrier substrate after each batch deposition to define a position-encoded carrier substrate;
d) determining a first spectral signature for each batch of functionalized plasmonic nanoparticles deposited on the position-encoded carrier substrate;
e) introducing a target molecule to the position-encoded carrier substrate and allowing the target molecule to bind to specific binding partners which interact with the target molecule, thereby inducing a shift in plasmon resonance from the first spectral signature to a second spectral signature; and
f) measuring the shift in the plasmon resonance from the first spectral signature to the second spectral signature.

19. A position-encoded sensor for plasmon resonance-based detection or quantification of binding affinities of a target molecule to a plurality of different binding partners, wherein the position-encoded sensor comprises:
a carrier substrate including a surface; and
a plurality of differently functionalized plasmonic nanoparticles randomly deposited on the surface, wherein the position of each differently functionalized plasmonic nanoparticle is recorded.

20. The position-encoded sensor according to claim 19, wherein the position of each differently functionalized plasmonic nanoparticle is recorded using dark field microscopy, reflection microscopy with crossed polarizers, total internal reflection microscopy or fluorescence microscopy.
